(19) United States
(12) Patent Application Publication
(10) Pub. No.: US 2010/0224562 A1
(43) Pub. Date: Sep. 9, 2010

(54) Ultraviolet Disinfection System and Method

(76) Inventors: Philip M. Rolchigo, Eden Prairie, MN (US); John H. Burban, Lake Elmo, MN (US); John Shanahan, White Bear Lake, MN (US); Robert O. Crowder, Lino Lakes, MN (US)

Correspondence Address: GREENBERG TRAURIG (PHX), INTELLECTUAL PROPERTY DEPARTMENT, 2450 COLORADO AVENUE , SUITE 400E, SANTA MONICA, CA, 90404, US

(21) Appl. No.: 12/398,785

(22) Filed: Mar. 5, 2009

(51) Int. Cl. C02F 1/32; A61L 2/10; B01D 61/58

(52) U.S. Cl. 210/650; 422/186.3; 210/243; 422/24; 210/652; 210/295

(57) ABSTRACT

Embodiments of the invention provide an ultraviolet disinfection system for use with a chamber containing fluid to be treated. The ultraviolet disinfection system includes an ultraviolet light source positioned outside of the chamber, a light manifold, and light fibers extending into the chamber. The light fibers radially disperse the ultraviolet light in order to provide a substantially uniform distribution of ultraviolet light along at least a portion of a longitudinal axis of the chamber.

### BACKGROUND

Ultraviolet (UV) light is used as an effective tool for water treatment. Conventional UV disinfection systems include a UV lamp housed in a water-tight quartz sleeve that is suspended in the water to be treated. This design, however, can pose problems in the areas of UV effectiveness, maintenance, and safety.

Due to heat generated by the UV lamps in conventional UV disinfection systems, minerals within the water supply scale on the quartz sleeves. Scaling on the quartz sleeves can reduce the intensity at which the UV light is emitted from the quartz sleeve and can reduce the effectiveness of the disinfection system. Fouled quartz sleeves therefore need to be periodically cleaned. Automatic cleaning systems can be expensive and are often ineffective. To remove the quartz sleeve for manual cleaning, the water supply system must be drained, which wastes energy and is impractical for many water supply applications. Also, UV disinfection systems operate most efficiently within a set temperature range. Applications where water is outside that temperature range require additional heating or cooling, thus consuming more power and requiring additional equipment.

### SUMMARY

Embodiments of the invention provide an ultraviolet disinfection system for use with a chamber containing fluid to be treated. Some embodiments of the ultraviolet disinfection system include an ultraviolet light source positioned outside of the chamber, a light manifold, and light fibers extending into the chamber. The light fibers radially disperse the ultraviolet light in order to provide a substantially uniform distribution 6f ultraviolet light along at least a portion of a longitudinal axis of the chamber.

### DESCRIPTION OF THE DRAWINGS

FIG. 1 is a cross-sectional view of an ultraviolet disinfection system according to one embodiment of the invention.

FIG. 2 is a cross-sectional view of an ultraviolet disinfection system according to another embodiment of the invention.

FIG. 3 is a cross-sectional view of an ultraviolet disinfection system according to another embodiment of the invention.

FIG. 4 is a cross-sectional view of an ultraviolet disinfection system according to another embodiment of the invention.

FIG. 5 is a cross-sectional view of an ultraviolet disinfection system according to another embodiment of the invention.

FIG. 6 is a cross-sectional view of an ultraviolet disinfection system according to another embodiment of the invention.

FIG. 7 is a cross-sectional view of an ultraviolet disinfection system according to another embodiment of the invention.

FIG. 8 is a perspective view of a core used in an ultraviolet disinfection system according to one embodiment of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

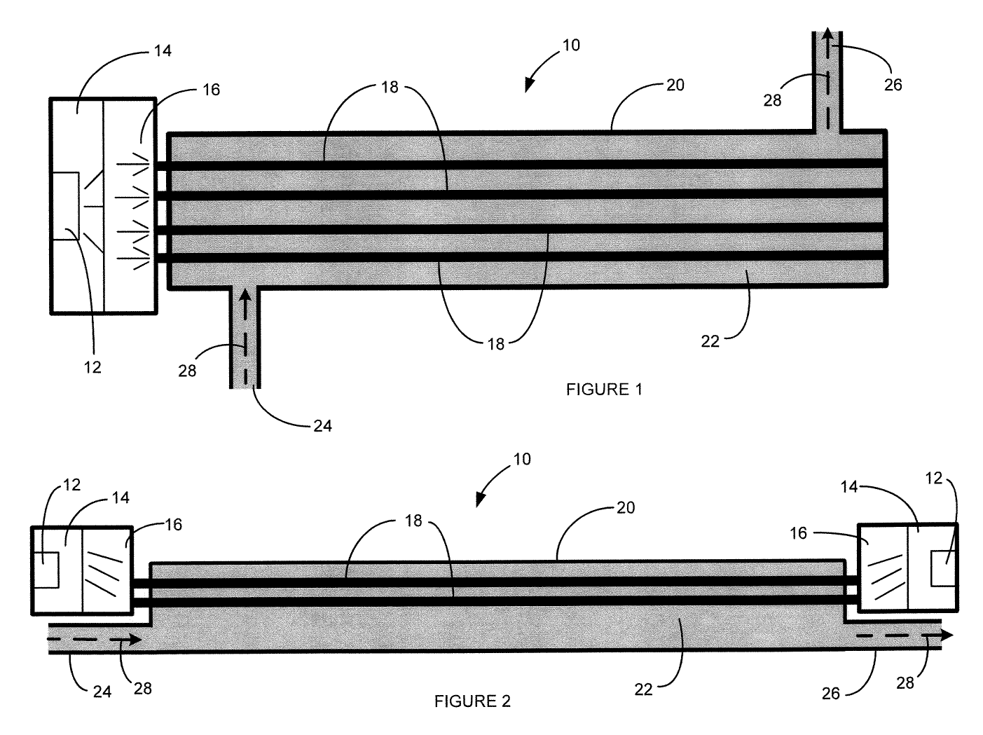

FIG. 1 illustrates an ultraviolet (UV) disinfection system 10 according to one embodiment of the invention. The UV disinfection system 10 can be used for the treatment of fluid (i.e., liquids and/or gases) in various applications, such as waste water applications, residential or municipal drinking water applications, industrial process water applications, swimming pools and water parks, aquariums, etc. The UV disinfection system 10 can include at least one UV light source 12, at least one lamp housing 14, at least one light manifold 16, and at least one light fiber 18.

As shown in FIG. 1, in some embodiments, the UV disinfection system 10 can be oriented laterally along a horizontal cylindrical chamber 20. The chamber 20 can be a retrofitted tube, an open vessel, a closed vessel, or a reservoir installed along a path of an existing filtration process, or the chamber 20 can be a tube or vessel specifically installed for UV disinfection, also known as a UV reactor. Fluid (e.g., water to be treated 22) can enter the chamber 20 at one end through an inlet 24 and can pass the entire length of the chamber 20 before exiting at another end through an outlet 26 (arrows 28 indicate fluid flow paths). In other embodiments, the UV disinfection system 10 can be oriented, either substantially vertically or horizontally, within a chamber 20 having any one of various sizes (e.g., the chamber 20 of FIG. 2). The chamber 20 can include a longitudinal axis.

In still other embodiments, the light fibers 18 can be suspended in an open chamber, as opposed to a flow-through reactor type of chamber 20. The light fibers 18 can be supported from either one of the ends or from both ends. The light fibers 18 can be suspended in a vertical or horizontal configuration.

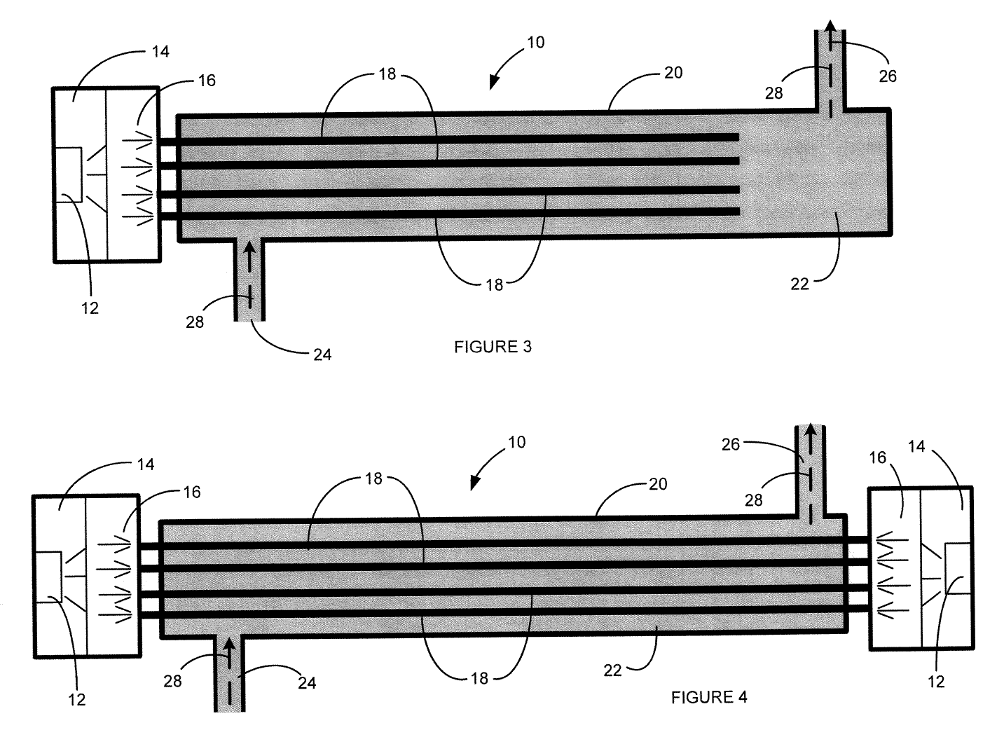

The UV light source 12 can be positioned within the lamp housing 14. The light manifold 16 can couple the lamp housing 14 to the light fibers 18. The light fibers 18 can extend into the chamber 20 and can extend along at least a portion of the length of the chamber 20, in some embodiments, as shown in FIGS. 2 and 3. In other embodiments, the light fiber 18 can extend along substantially the entire length of the chamber 20, as shown in FIG. 1. UV light is emitted from the UV light source 12 in the lamp housing 14. The light manifold 16 can direct the UV light into the light fibers 18. The light fibers 18 can substantially uniformly disperse the UV light along their lengths to irradiate the fluid as it flows through the chamber 20. In some embodiments, the UV disinfection system 10 can also include a monitoring and/or control system with power sensors, UV intensity sensors, flow rate sensors, temperature sensors, and/or turbidity sensors (not shown) to monitor and validate proper operation.

In some embodiments, a single light manifold 16 can be used to direct the UV light into the light fibers 18. In other embodiments, as shown in FIGS. 2 and 4, the light fibers 18 can extend between a first UV light source 12 and a second UV light source 12. The second UV light source 12 can be positioned at a second location adjacent to the chamber 20, for example, at an end of the chamber 20 opposite to the first UV light source 12. The light fibers 18 can be secured at both ends between the two separate light manifolds 16.

In general, the UV disinfection system 10 can include multiple light manifolds 16 attached to multiple light fibers 18, where each light fiber 18 extends along at least a portion of the length of the chamber 20. In some embodiments, multiple light sources 12 of different types can be used in the light manifolds 16. All of the light sources 12 can be turned on and can operate at the same time, or some of the light sources 12 can be turned on while others are turned off. The number and type of light sources 12 turned on or off can be dictated by the various sensors included in the UV disinfection system 10 in order to ensure effective disinfection despite variations in water quality.

The UV light source 12 can be any type of source that is able to produce UV light at germicidal wavelengths (e.g., between about 150 nanometers and about 300 nanometers, in the UV-C range, and in one embodiment, about 254 nanometers). In some embodiments, the UV light source 12 can be a low or medium pressure, high output mercury lamp. The UV light source 12 can be positioned outside of the chamber 20 and not submerged in the fluid. This outside arrangement can allow the UV light source 12 to operate more efficiently compared to systems where a UV light source is submerged in the fluid, because the UV light source 12 can operate within its optimum temperature range without requiring temperature control of the fluid. Operating at the optimum range can also lengthen the life of the UV light source 12, as well as improve its power efficiency. In addition, as heat from the UV light source 12 is emitted outside the chamber 20, the UV light that is introduced into the chamber 20 contains much less heat. This can prevent scaling of minerals on the light fibers 18, as seen on hot quartz sleeves of conventional submerged UV light sources. Also, this outside arrangement makes lamp maintenance much easier, because none of the components need to be removed from the chamber 20 to replace the UV light source 12. If the UV light source 12 were to break, there is no risk of mercury exposure in the fluid.

The light fibers 18 can be waveguides or optical fibers that are able to transmit UV light from the UV light source 12 into the chamber 20, and in some embodiments, without substantial losses in intensity. The light fibers 18 can disperse the UV light to provide a substantially uniform distribution of UV light along at least a portion of the longitudinal axis of the chamber 20. In some embodiments where the light fibers 18 are optical fibers, the light fibers 18 include a core surrounded by a cladding layer. The UV light can be transmitted through the core and can “leak” through the cladding at certain points to provide a uniform UV light distribution along the length of the light fibers 18. A variety of techniques can be used to provide cladding leakage, including removal of portions of cladding or manufacturing or printing the light fibers 18 with patterns of cladding. The end or other portions of the light fibers 18 can also include a diffuser (not shown) to further spread the UV light.

The substantially uniform distribution of UV light can prevent one area within the chamber 20 from being over-intensified, while other areas receive a relatively low intensity UV light. This configuration allows the fluid to receive substantially constant UV light intensity over a sufficient length of time (i.e., the time it takes the fluid to flow the length of the light fibers 18). With the correct disinfection time and UV light intensity, the fluid can receive the proper UV dose (the product of time and intensity) to effectively deactivate and/or eliminate microorganisms, such as Giardia and cryptosporidium. If the UV light were only to project in a limited area, the intensity would need to be much higher to provide the proper UV dose, because the time of UV treatment would be greatly reduced. Also, in conventional systems with short UV disinfection times, some microorganisms can be adsorbed into solids in the fluid, can clump together, or can be hidden behind larger microorganisms or solids that absorb the UV light in their path. These microorganisms are then temporarily shielded from the UV light and can still replicate after passing the UV light source. Having an extended flow path in substantially constant presence of UV light can help prevent this shielding issue.

In some embodiments, reflective surfaces (not shown) can be positioned at various positions within the chamber 20 to allow the UV light to reflect back toward the fluid, rather than being absorbed by the internal walls of the chamber 20. This can increase the UV dose without requiring an increase in intensity of the UV light emitted from the UV light source 12. The reflective surfaces can also be used to provide substantially equal UV light intensity levels throughout the chamber 20, even though UV light is provided in a variety of locations, including asymmetric or irregular patterns.

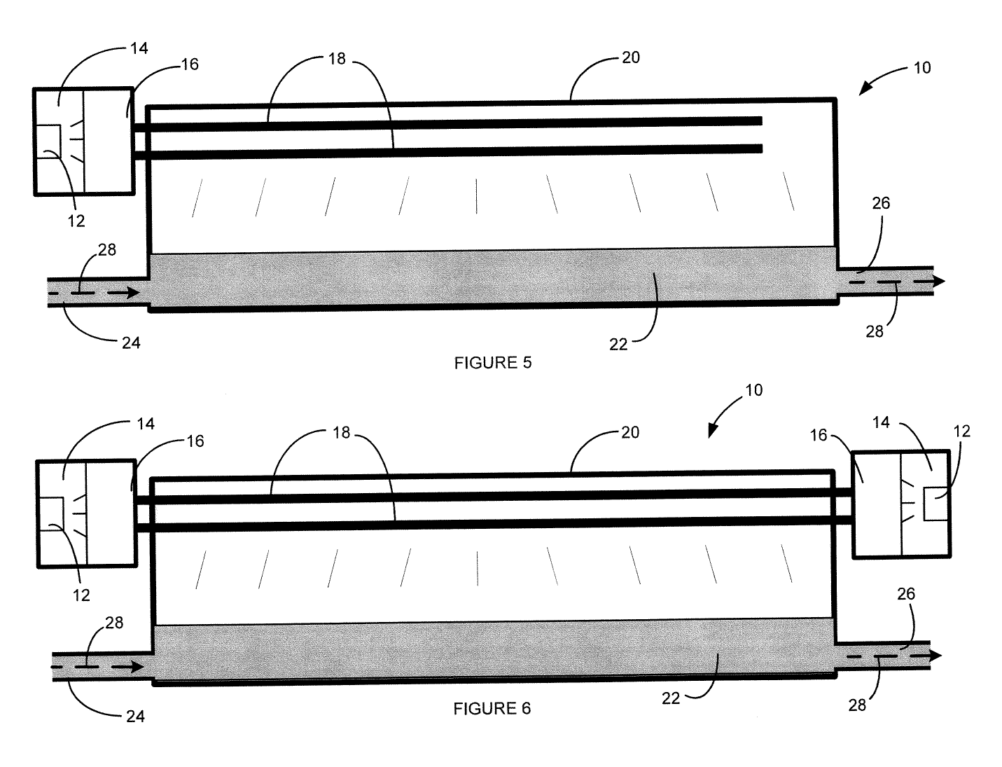

In some embodiments, the light fibers 18 can be configured to disperse light radially (e.g., up to about 360 degrees around the longitudinal axis of the light fibers 18). In other embodiments, the light fibers 18 can be configured to emit light in particular directions (e.g., less than 360 degrees around the longitudinal axis of the light fibers 18). For example, as shown in FIGS. 5 and 6, the chamber 20 can be configured to provide a fluid path with an air cavity above the fluid path. The light fibers 18 can be positioned above the fluid path to emit UV light only downward toward the fluid path (e.g., up to about 180 degrees around the longitudinal axis of the light fibers 18). In addition, rather than the rigid, tubular mercury lamps of conventional systems, the light fibers 18 can be flexible and/or curved to carry the UV light from the UV light source 12 along various lengths and to irradiate places that would be unreachable with conventional systems.

Certain characteristics of the UV disinfection system 10 can be configured for the type of application in which it will be used. For example, different microorganisms have different optimal UV doses for deactivation. As a result, some applications have higher or lower UV dose requirements depending on the target microorganisms. Those applications that have higher UV dose requirements can use a higher UV intensity, longer light fibers 18 for a longer disinfection time, more reflective surfaces in the chamber 20, etc.

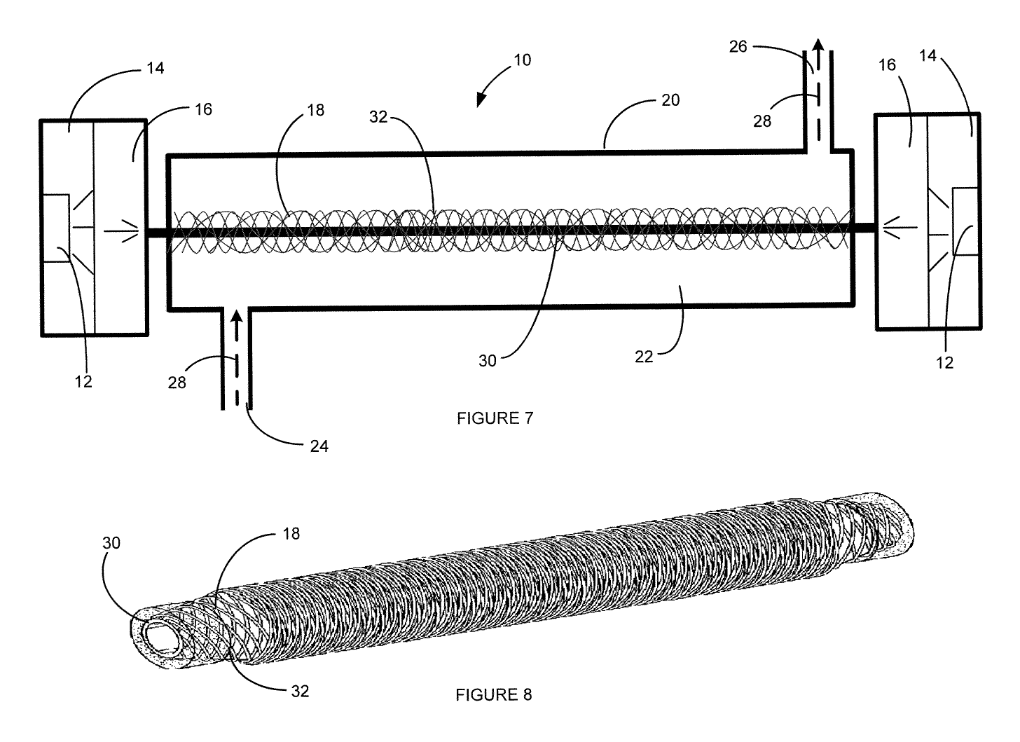

As shown in FIGS. 7 and 8, in some embodiments, multiple light fibers 18 can be helically wound around a core 30, creating a helix of light fibers 18 along at least a portion of the length of the chamber 20. In some embodiments, photocatalyst fibers 32, such as fibers coated with titanium dioxide, can also be helically wound around the core 30. The light fibers 18 and the photocatalyst fibers 32 can be helically wound together around the core 30, either uniformly or randomly. In other embodiments, the photocatalyst fibers 32 are not helically wound, but rather are substantially straight and are positioned adjacent to light fibers 18 that are also substantially straight in the chamber 20.

In the presence of the UV light, the titanium dioxide or other photocatalyst can react with nearby water molecules to create highly reactive hydroxyl radicals. The hydroxyl radicals can react with the organic compounds, such as microorganisms within the water, and break them down into water and carbon dioxide, thus helping to further purify the water. The photocatalyst fibers 32 can improve the efficiency of the UV disinfection system 10. In the configuration shown in FIG. 7, the fluid to be treated can flow substantially perpendicular to the photocatalyst fibers 32, thus improving mass transfer of microorganisms through the helically wound light fibers 18 and the photocatalyst fibers 32 in order to increase the probability of reactions between the microorganisms and the photocatalyst.

The UV disinfection system 10 can provide improved performance for systems having intermittent flow. In a conventional UV system, if fluid flow stops, heat builds up. This build up of heat reduces the life of the UV bulb. If a temperature sensor is employed, the temperature sensor may sense an over-temperature condition and shut off energy to the UV bulb. When fluid starts flowing again, the UV bulb will be re-energized, but the UV bulb will not instantly reach the proper operating conditions. As a result, the UV light being delivered will not be at the proper intensity to disinfect the fluid. In some embodiments of the invention, heat management is performed externally from the fluid to be treated. Heat management can be independent of fluid conditions, such as temperature and fluid flow rate. In some embodiments, when fluid stops flowing, the UV light source 12 remains energized and the UV disinfection system 10 irradiates the same volume of fluid for a longer period of time. When the fluid begins flowing again, the UV light source 12 has remained energized and continues to deliver the proper intensity of UV light to disinfect the fluid. Accordingly, the UV disinfection system 10 provides external heat management of the UV light source 12 that is independent of the conditions of the fluid to be treated. Additionally, the temperature of the fluid is not increased by the UV light source, enabling better control of fluid temperatures.

The UV disinfection system 10 can be positioned in a water treatment system where fluid disinfection is needed, such as at the end of a water treatment process. The UV disinfection system 10 can also be used in conjunction with other disinfection or purification processes, such as a reverse osmosis (RO) process or an ultrafiltration process. In some embodiments, the UV disinfection system 10 can be used at a stage in the water treatment process before the RO process to prevent organisms from fouling the RO membranes. In other embodiments, the UV disinfection system 10 can be used before and after the RO process or only after the RO process.

In some embodiments, the UV disinfection system 10 can be cleaned using an isolated cleaning loop. The isolated cleaning loop can be used to clean the light fibers 18 while the light fibers 18 and the other components of the UV disinfection system 10 remain in place. In addition, the isolated cleaning loop can clean the light fibers 18 while the fluid to be treated 22 remains inside the chamber 20. During cleaning, the cleaning loop can be engaged and isolated from the inlet and outlet fluid ports via isolation valves. The cleaning loop can include a pump to re-circulate water or water and cleaning chemicals past the fibers. Chemical addition pumps can be connected to the cleaning loop to adjust the pH in the cleaning loop and/or to add chemical oxidants to the loop. A discharge port on the isolation loop can be included to allow flushing of the cleaning loop and the fibers with water before returning to operation. The valves and pumps within the cleaning loop can be activated manually, automatically by an onboard controller and based upon a cleaning schedule, or automatically by an onboard controller in response to inputs from onboard sensors.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. An ultraviolet disinfection system for use with a chamber containing fluid to be treated, the chamber having a longitudinal axis, the system comprising:
an ultraviolet light source positioned outside of the chamber, the ultraviolet light source emitting ultraviolet light;
a light manifold in communication with the ultraviolet light source; and
at least one light fiber in communication with the light manifold, the at least one light fiber extending into the chamber, the at least one light fiber radially dispersing the ultraviolet light in order to provide a substantially uniform distribution of ultraviolet light along at least a portion of the longitudinal axis of the chamber.

2. The system of claim 1 wherein the at least one light fiber includes at least one optical fiber.

3. The system of claim 1 wherein the at least one light fiber includes a core and cladding surrounding the core, and wherein the cladding includes leaks and the ultraviolet light is distributed from the leaks.

4. The system of claim 1 wherein substantially an entire length of the at least one light fiber is submerged in the fluid to be treated.

5. The system of claim 1 and further comprising a diffuser coupled to the at least one light fiber.

6. The system of claim 1 and further comprising at least one of a temperature sensor, a flow sensor, a power sensor, an ultraviolet intensity sensor, and a turbidity sensor.

7. The system of claim 1 wherein the ultraviolet light source is a low pressure, high output mercury lamp.

8. The system of claim 1 wherein the ultraviolet light source is a medium pressure, high output mercury lamp.

9. The system of claim 1 wherein a wavelength of the ultraviolet light is between about 150 nanometers and about 300 nanometers.

10. The system of claim 1 wherein a wavelength of the ultraviolet light is in a range of UV-C wavelengths.

11. The system of claim 1 and further comprising reflective surfaces coupled to an internal portion of the chamber in order to reflect the ultraviolet light.

12. The system of claim 1 wherein the chamber includes an inlet and an outlet, the fluid flows from the inlet to the outlet creating a flow path, and the flow path is directed at least one of perpendicular to the at least one light fiber and parallel to the at least one light fiber.

13. The system of claim 1 and further comprising a second ultraviolet light source positioned outside of the chamber and a second light manifold.

14. The system of claim 1 wherein the at least one light fiber includes a plurality of helically wound light fibers.

15. The system of claim 14 wherein fluid flows substantially perpendicular to a longitudinal length of the plurality of helically wound light fibers.

16. The system of claim 1 and further comprising at least one photocatalyst fiber extending into the chamber.

17. The system of claim 16 wherein the at least one photocatalyst fiber includes titanium dioxide.

18. The system of claim 1 and further comprising a plurality of light fibers and a plurality of photocatalyst fibers helically wound together and extending into the chamber.

19. The system of claim 18 wherein fluid flows substantially perpendicular to a longitudinal length of the plurality of light fibers and the plurality of photocatalyst fibers helically wound together.

20. The system of claim 1 wherein the ultraviolet disinfection system is used in conjunction with at least one of a reverse osmosis process and an ultrafiltration process.

21. The system of claim 1 wherein the ultraviolet disinfection system uses external heat management that is independent of conditions of the fluid to be treated.

22. A method of treating a fluid in a chamber, the chamber having a longitudinal axis, the method comprising:
providing an ultraviolet light source outside of the chamber;
directing ultraviolet light from the ultraviolet light source to a light manifold;
directing the ultraviolet light from the light manifold to at least one light fiber extending inside the chamber; and
radially dispersing the ultraviolet light through the at least one light fiber in order to substantially uniformly distribute ultraviolet light along at least a portion of the longitudinal length of the chamber.

23. The method of claim 22 and further comprising cladding the at least one light fiber and allowing ultraviolet light to leak from the cladding.

24. The method of claim 22 and further comprising submerging substantially an entire length of the at least one light fiber in the fluid to be treated.

25. The method of claim 22 and further comprising monitoring at least one of temperature, flow, power, ultraviolet intensity, and turbidity.

26. The method of claim 22 and further comprising providing ultraviolet light having a wavelength between about 150 nanometers and about 300 nanometers.

27. The method of claim 22 and further comprising providing ultraviolet light having a wavelength in a range of UV-C wavelengths.

28. The method of claim 22 and further comprising reflecting ultraviolet light inside the chamber.

29. The method of claim 22 and further comprising flowing fluid to be treated substantially perpendicular to the at least one light fiber.

30. The method of claim 22 and further comprising providing a second ultraviolet light source positioned outside of the chamber and a second light manifold.

31. The method of claim 22 and further comprising providing a plurality of helically wound light fibers extending inside the chamber.

32. The method of claim 22 and further comprising providing at least one photocatalyst fiber extending inside the chamber.

33. The method of claim 22 and further comprising providing at least one photocatalyst fiber including titanium dioxide.

34. The method of claim 22 and further comprising providing a plurality of light fibers and a plurality of photocatalyst fibers helically wound together and extending inside the chamber.

35. The method of claim 22 and further comprising treating the fluid with at least one of a reverse osmosis process and an ultrafiltration process.

36. The method of claim 22 and further comprising managing heat production independent of conditions of the fluid to be treated.
